(19) United States
(12) Patent Application Publication
(10) Pub. No.: US 2015/0012109 A1
(43) Pub. Date: Jan. 8, 2015

(54) CUP FOR AN ORTHOPAEDIC IMPLANT, ORTHOPAEDIC IMPLANT COMPRISING SUCH A CUP AND METHOD FOR PRODUCING SUCH A CUP

(71) Applicant: GALACTIC, Rochefort-du-Gard (FR)

(72) Inventors: Pierre-Etienne Moreau, Montaud (FR); Thibault Loriot De Rouvray, St Cyr Sur Loire (FR); Thomas Brosset, Les Taillades (FR); Julien Dumas, Nice (FR); Jean-Michel Delobelle, Saint Saulve (FR); Henri-Paul Prudent, Comps (FR); Jean-François Bataille, Rochefort Du Gard (FR); Bertrand Blandet, Boulbon (FR)

(21) Appl. No.: 14/378,967

(22) PCT Filed: Feb. 19, 2013
(86) PCT No.: PCT/FR2013/050335

§ 371 Date: Aug. 14, 2014

(30) Foreign Application Priority Data
Feb. 20, 2012 (FR) 1251515

(51) Int. Cl. A61F 2/30; B29C 67/00

(52) U.S. Cl. CPC A61F 2/3094; B29C 67/0077; A61F 2002/30968; A61F 2240/00
USPC 623/23.43; 264/497

(57) ABSTRACT

A cup having an inner cavity, for an articulation organ, and a metallic outer layer and in a portion of a spheroid, the outer layer including networks of meshes with nodes and struts, where the struts are tapered struts each having a tapered shape and being arranged such that the tapered shapes are uniformly oriented.

### TECHNICAL FIELD

The present invention relates to a cup for an orthopedic implant. In addition, the present invention relates to an orthopedic implant, such as a cotyloidal implant, comprising such a cup. Furthermore, the present invention relates to a method for manufacturing such a cup.

### BACKGROUND

The present invention finds application in particular in the field of reconstructive surgery and orthopedics. In particular, the present invention finds application in the production of cups for orthopedic implants to be implanted in the acetabular cavity, so as to form a hip prosthesis.

EP0149975A1 describes a cup for a cotyloidal implant having an inner cavity adapted to accommodate an articulation organ, such as a prosthetic femoral head. The cup has an outer layer which has overall the shape of a semi-spheroid and which is intended to be secured to the iliac bone. For this purpose, the outer layer is striated with grooves on which the bone tissue may grip and grow on.

However, the cup of EP0149975A1 is provided for being permanently implanted in the iliac bone. However, it is sometimes necessary to extract the hip prosthesis, for example to replace a faulty component of the hip prosthesis. But the extraction of a cup of the prior art involves a random tearing with forces in all directions, which could destroy a significant amount of bone tissue surrounding the cup.

Furthermore, the cup of EP0149975A1 has an outer surface of relatively small surface area, thus limiting the growth and gripping of the bone tissue on the cup.

### BRIEF SUMMARY

The present invention aims in particular to solve, totally or partly, the problems mentioned hereinbefore.

To this end, the invention relates to a cup, for an orthopedic implant such as a cotyloidal implant, intended to be implanted in a bone, the cup having an inner cavity adapted to accommodate an articulation organ, the cup having an outer layer intended to be secured to the bone, the outer layer having overall the shape of a spheroidal portion, preferably the shape of a semi-spheroid, the outer layer being made of metallic material;

the cup being characterized in that the outer layer comprises at least a network of meshes defined by nodes and struts connecting the nodes together, each node being formed by the intersection of several struts, said struts comprising struts called tapered struts each having a tapered shape, said tapered struts being arranged such that the tapered shapes are uniformly oriented.

In other words, the outer layer is porous and the envelop of the orientation directions of all the tapered shapes is a spheroid portion.

Thus, such a cup makes it possible to perform a local ablation of bone tissue, as opposed to an overall tearing. To this end, the operator can indeed impart a determined movement to the orthopedic implant, and therefore to the cup. For example, this determined movement may be a revolution with a left-hand pitch aligned with the direction of orientation of the tapered shapes. The tapered shapes thus cut the bone tissue. When the operator extracts the cup, the extraction force to be exerted is relatively low and damage of the bone tissue is reduced to what is strictly necessary. Moreover, such a mesh network supports the growth of bone tissue and its good gripping on the outer layer.

Throughout the present application, the term “network” refers to a set of nodes having at least two dimensions and having a spatial periodicity, that is to say when we translate in space according to certain vectors, we find exactly the same environment.

Throughout the present application, the term “tapered shape” refers to a relatively thin and elongated shape and that tapers towards at least one of its edges. For example, a knife blade has a tapered shape. In other words, a tapered shape has at least one edge called sharp edge which has a radius of curvature smaller than the radius of curvature of another edge of the tapered shape. Typically, the radius of curvature of a sharp edge can be ranging from 0.1 mm to 0.15 mm, while the radius of curvature of another edge can be ranging from 0.15 mm to 0.5 mm.

Throughout the present application, the term “uniform” indicates that the tapered shapes have a common general orientation which extends parallel to the spheroidal surface of the outer layer.

In practice, the outer layer may be in the shape of a sphere or in the shape of a spheroid flattened at the poles and enlarged at the equator, in the manner of a geoid.

According to an embodiment of the invention, said tapered struts are arranged such that their tapered shapes are oriented along respective directions which are circumferential directions for the portion of the spheroid.

In other words, a circumferential direction is a direction locally tangent to the portion of the spheroid. Thus, such tapered shapes allow efficient ablation of the bone tissue by a revolution movement of the cup around a determined axis.

According to an embodiment of the invention, at least a subset of tapered struts has tapered shapes oriented along a direction parallel to the equatorial plane of the portion of the spheroid.

Thus, such a subset of tapered struts allows efficient ablation of the bone tissue by a revolution movement of the orthopedic cup around the polar axis of the portion of the spheroid.

In the present application, the term “subset” refers to a plurality of struts that are oriented according to at least one common orientation axis. Typically, the struts of a subset can be parallel to each other; they can have an axis parallel to a determined direction which is tangent to the portion of the spheroid.

The outer layer can comprise one subset or subsets of tapered struts that can participate in the ablation of bone tissue, as well as subsets of non-tapered struts, for example round, which do not participate in the ablation of bone tissue. The ablation of bone tissue is carried out along a preferential direction that is defined by the subset(s) of tapered struts.

According to an embodiment of the invention, at least two tapered struts converge at each node.

Thus, the presence of several tapered struts at each node allows achieving an effective ablation of the bone.

According to a variant of the invention, at least one subset of struts has its tapered shapes oriented along a direction perpendicular to the equatorial plane of the portion of the spheroid.

Thus, such a subset of struts allows an efficient ablation of bone tissue by a translational movement of the cup towards its equatorial plane and out of the acetabular cavity.

According to a variant of the invention, at least one subset of struts has its tapered shapes oriented along directions forming a 45° angle with the equatorial plane of the portion of the spheroid.

Thus, such a subset of struts allows an efficient ablation of bone tissue by a revolution movement of the cup around an instant center of rotation offset to the portion of spheroid.

According to an embodiment of the invention, each strut has overall the shape of a cylinder the axis of which connects two consecutive nodes of said at least one network.

Thus, such a cylinder shape ensures a homogeneous ablation over the entire length of a strut. The ablation of the bone tissue is therefore carried out at any point of the network or each network, which further facilitates the extraction of the cup, and therefore of the orthopedic implant.

According to an embodiment of the invention, each tapered strut has a cross-section that is oblong and symmetrical with respect to its longitudinal axis, each tapered strut preferably having an overall pear-shaped cross-section.

Thus, such an oblong and symmetrical cross-section relatively simplifies the manufacture of the struts.

According to a variant of the invention, the tapered shape has two edges that are relatively “sharp” or have relatively small radius of curvature.

In other words, each tapered shape has two cutting edges on the same blade. Thus, the movement of ablation may be achieved indifferently in both directions of the uniform orientation of the tapered shapes.

According to an embodiment of the invention, the outer layer comprises several juxtaposed networks, meshes respectively belonging to two consecutive networks forming a dihedral angle of less than 35°, preferably less than 25°.

Thus, such juxtaposed networks simplify the manufacture of the cup covered, completely or almost completely, with network(s), because the struts of a network may have constant directions not necessarily linked to radial, axial or circumferential directions of the portion of the spheroid.

According to an embodiment of the invention, each network substantially covers a quarter of the portion of the spheroid, each quarter extending between meridians spaced apart by an angle of less than 35°, preferably less than 25°.

Thus, such network parts by quarter allow maling a cup of which the networks generally have a spheroidal shape, that is to say of which the struts extend along directions approaching the radial, axial or circumferential directions of the portion of the spheroid.

According to an embodiment of the invention, the outer layer comprises two mesh networks which are interpenetrating and the meshes of which have equivalent dimensions.

In other words, the meshes are stacked and shifted with identical orientations between meshes of the interpenetrating networks. By analogy with the crystalline structures, such interpenetrating networks could be described as an “body-centered cubic” arrangement.

Thus, such interpenetrating networks allow increasing the dimensions of the porosities of the outer layer with respect to the section of the struts, which makes it possible to create an outer layer having a higher porosity, therefore better suited to the growth of bone tissue.

According to an embodiment of the invention, each mesh has dimensions ranging between 200 micrometers and 800 micrometers, preferably between 430 micrometers and 650 micrometers.

Thus, such dimensions of each mesh support a proper growth of the bone tissue. The dimensions of the meshes indeed define porosities or void volumes, in which the bone tissue can develop.

According to an embodiment of the invention, each mesh has an overall parallelepiped shape with a rectangular base, preferably right parallelepiped shape, each mesh having for example a cube shape.

Thus, such mesh geometry is relatively simple to make.

According to an embodiment of the invention, the density of the outer layer is ranging between 30% and 90%, preferably between 60% and 80%, yet preferably equal to about 75%.

Thus, such a density offers a high porosity, which allows a rapid and dense growth of the bone tissue.

According to an embodiment of the invention, the outer layer has a thickness ranging between 0.3 mm and 7 mm, preferably between 0.5 mm and 3 mm.

Thus, such a thickness of the outer layer allows a high cohesion with the bone tissue.

According to a variant of the invention, the outer layer occupies 80% of the height of the cup, which further increases the cohesion of the bone tissue.

According to an embodiment of the invention, the metallic material is a material that is biocompatible, implantable and compatible with a generative method by powder sintering, the metallic material can in particular be selected from the group consisting of pure titanium, a titanium, chromium, cobalt, and stainless steel based alloy.

Thus, such a metallic material confers to the outer layer and to the cup the mechanical and chemical resistance necessary to its bone implantation. Furthermore, such a metallic material may be implemented in a generative method, in order to achieve a cup in accordance with the invention.

Furthermore, the present invention relates to an orthopedic implant comprising a cup according to the invention and an articulation organ formed by an insert attached within the inner cavity, for example by press-fitting.

In other words, such an orthopedic implant comprises two main components, of which an insert which forms the inner cavity for receiving a prosthetic femoral head.

Furthermore, the present invention relates to a method, for making a cup according to the invention, the method comprising the steps of:
forming a powder stratum of the metallic material;
implementing a generative method machine, for example a selective laser sintering machine, so as to sinter the stratum in a determined manner by a control unit;
repeating the aforementioned steps until the cup is formed.

Thus, such a method allows making a cup in accordance with the invention, with a particularly high accuracy.

The embodiments and the variants mentioned hereinabove may be taken separately or according to any technically permissible combination.

### BRIEF DESCRIPTION OF THE DRAWINGS

The present invention will be well understood and its advantages will also appear in the light of the following description, given solely by way of non-limiting example and made with reference to the accompanying drawings, wherein:

FIG. 1 is a perspective view of a cup in accordance with the invention;

FIG. 2 is a perspective view, truncated by a meridian plane II in FIG. 1, of the cup of FIG. 1;

FIG. 3 is a view similar to FIG. 2, at an angle different from that of FIG. 2;

FIG. 4 is a sectional view, along the plane IV in FIG. 3, of the cup of FIG. 3;

FIG. 5 is a sectional view of an orthopedic implant in accordance with the invention comprising the cup of FIG. 4;

FIG. 6 is a view at a larger scale of a part of the cup of FIG. 2;

FIG. 7 is a view at a larger scale of a part of the cup of FIG. 6;

FIG. 8 is a view at a larger scale of the detail VIII in FIG. 2;

FIG. 9 is a view at a larger scale of a part of the cup of FIG. 7;

FIG. 10 is a view similar to FIG. 9 of a part of a cup in accordance with a second embodiment of the invention; and

FIG. 11 is a view similar to FIG. 10 illustrating at a smaller scale the part of FIG. 10.

### DETAILED DESCRIPTION

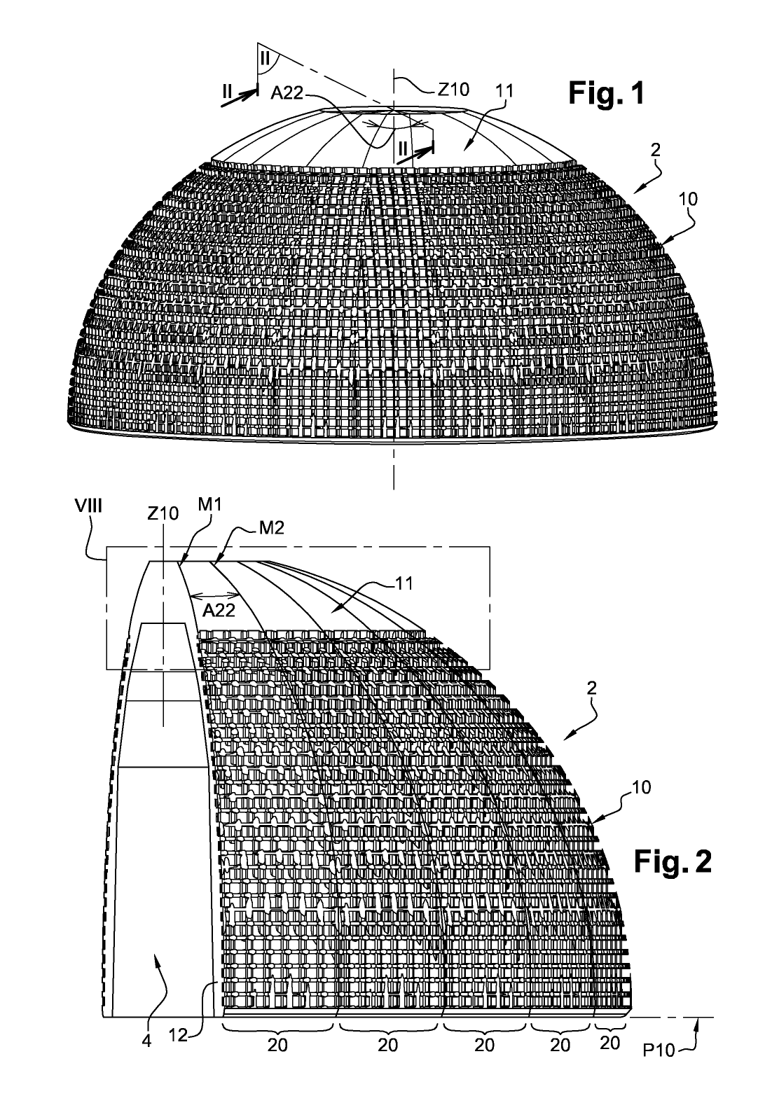

FIG. 1 illustrates a cup 2 in accordance with the invention, to form an orthopedic implant 1 according to the invention and visible in FIG. 5. The cup 2 is intended to be implanted in an iliac bone at the location of an acetabular cavity, not shown.

As shown in FIG. 2, the cup 2 has an inner cavity 4 adapted to accommodate an articulation organ of the orthopedic implant 1, as described hereinafter in connection with FIG. 5.

The cup 2 has an outer layer 10 intended to be secured to the iliac bone, not shown. The outer layer 10 has overall the shape of a semi-spheroid, of a polar axis Z10 and equatorial plane P10. Throughout the present application, the term “outer” is used as opposed to the term “inner”. The outer layer 10 therefore has a position opposite to the inner cavity 4.

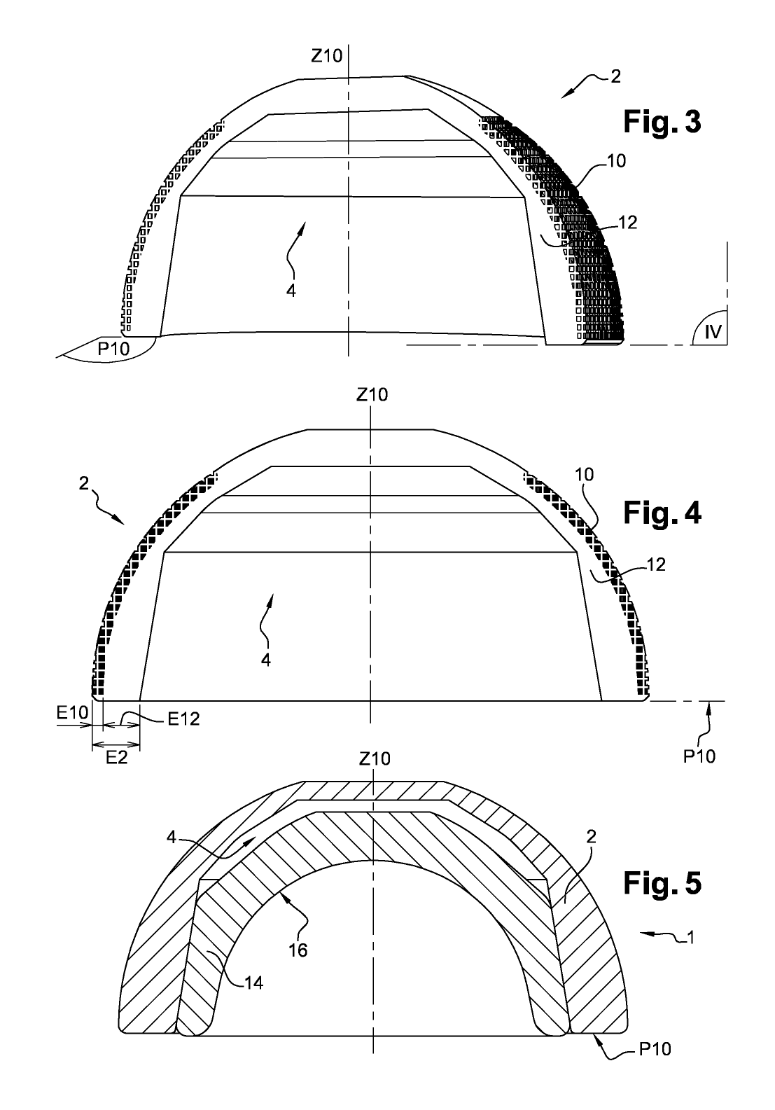

As shown in FIGS. 3 and 4, the cup 2 also has an inner layer 12 that defines the inner cavity 4. In the example of FIGS. 1 to 4, the outer layer 10 has a thickness E10 of about 1 mm and the inner layer 12 has a thickness E12 of about 4 mm. As the thicknesses E10 and E12 vary depending on the latitude on the outer layer 10, the thicknesses E10 and E12 are measured herein at the equatorial plane P10 of the semi-spheroid forming the outer layer 10. At this location, the outer layer 10 represents about 16% of the thickness E2 of the cup 2.

The outer layer 10 is made of metallic material. Similarly, the inner layer 12 is made of metallic material, which in this case is similar to that forming the outer layer 10. Indeed, in the example of FIGS. 1, 2, 3 and 4, the outer layer 10 and the inner layer 12 are made integral, as a single-piece. The metallic material herein is a titanium, chromium, cobalt based alloy, as defined for example by the ISO 5832 and ASTM F136 standards. This metallic material is biocompatible, implantable and compatible with a generative method by powder sintering.

FIG. 5 illustrates the orthopedic implant 1 in accordance with the invention which comprises the cup 2 and an articulation organ formed by an insert 14 which is attached within the inner cavity 4 by press-fitting. The insert 14 has an inner articulation surface 16 which is substantially spherical for receiving a prosthetic femoral head, not shown. In the example of FIG. 5, the orthopedic implant 1 is a cotyloidal implant for a hip prosthesis.

As shown in FIG. 2, the outer layer 10 comprises several mesh networks, of which five are visible in FIG. 2 with the reference number 20. In the example of FIGS. 1 to 4, the networks 20 cover a substantial part of the outer layer 10. There remains a spherical cap 11 not covered by the networks 20. The spherical cap 11 represents herein about 20% of the surface area of the semi-spheroid and the outer layer 10 represents herein about 80% of the surface area of the semi-spheroid. In the example of FIG. 2, the outer layer 10 extends over about 80% of the height of the semi-spheroid.

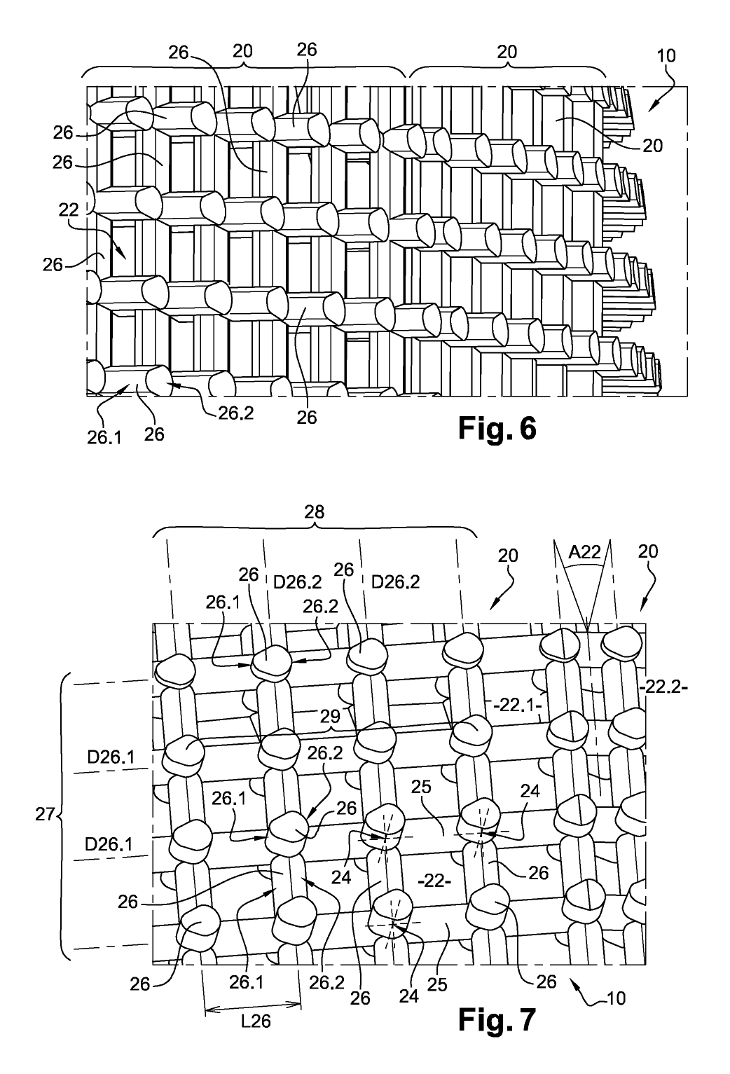

As shown in FIGS. 6 and 7, each network 20 comprises meshes 22. Each mesh 22 is defined by nodes 24 and by struts 25 and 26 connecting the nodes 24 together. Each node 24 is formed by the intersection of several struts 25 and 26. The struts 25 and 26 comprise struts called tapered struts 26 each having a tapered shape. In addition, the struts 25 and 26 comprise struts 25 each having overall a cylindrical shape with a circular base and with an axis perpendicular to the respective axes of the tapered struts 26.

In the example of FIG. 7, out of three struts intersecting or converging at a respective node 24, two struts 26 are tapered struts 26, the third strut, strut 25, has overall a cylindrical shape with a circular base and with an axis perpendicular to the respective axes of the tapered struts 26.

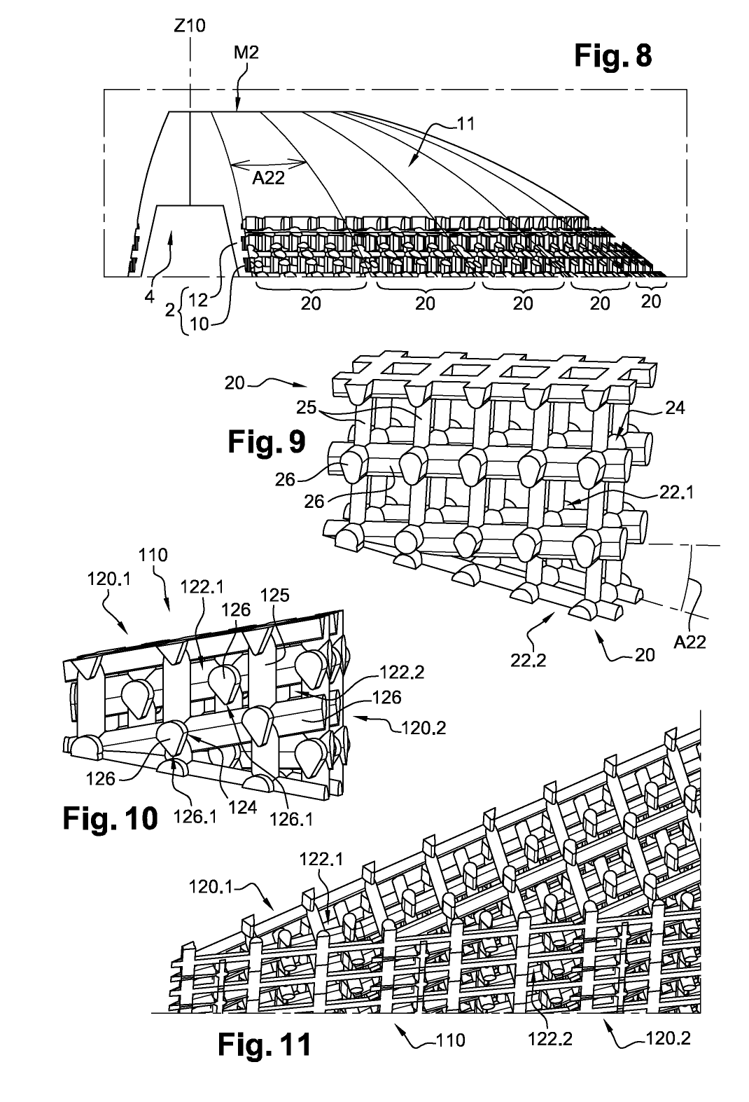

In the example of FIGS. 1 to 8, each mesh 22 has overall a cubic shape. For this purpose, in each network 20, a subset 27 of struts 25 is perpendicular to two subsets 28 and 29 of struts 26. The subset 27 comprises struts 25 parallel to each other, the subset 28 comprises tapered struts 26 parallel to each other and the subset 29 comprises tapered struts 26 parallel to each other.

In the example of FIGS. 1 to 9, the struts 26 of the subset 28 are locally parallel to each other. The struts 25 of the subset 27 are locally parallel to each other. The struts 26 of the subset 29 are locally parallel to each other.

In practice, each mesh 22 has dimensions L26 which are identical and measuring about 600 micrometers. The density of the outer layer 10 is about 75%. The density of the outer layer 10 is calculated by performing the ratio having:
as numerator, the volume of material of the outer layer 10 comprising the networks 20; in other words, the “real” volume of the outer layer 10; and
as denominator, the volume geometrically delimited by the envelop of the outer layer 10 considered as solid, in other words the “virtual” volume of the outer layer 10.

As shown in FIGS. 6 and 7, each tapered strut 26 has overall a tapered shape. Thus, each tapered strut 26 has a sharp edge 26.1 which has a radius of curvature smaller than the radius of curvature of another edge 26.2 of the tapered strut 26. The radius of curvature of a sharp edge 26.1 is about 0.10 mm, while the radius of curvature of another edge 26.2 is about 0.15 mm. A sharp edge 26.1 corresponds to the “cutting edge” of a tapered strut 26.

The tapered struts 26 are arranged such that the tapered shapes, which taper towards the sharp edges 26.1, are uniformly oriented. In other words, the tapered shapes of the tapered struts 26 have a common general orientation which extends parallel to the semi-spheroid forming the outer layer 10.

In the example of FIGS. 6 and 7, the cylindrical struts 25 and the tapered struts 26 respectively belonging to the subsets 27 and 28 are oriented along respective directions D26.1 and D26.2. The respective directions D26.1 and D26.2 are circumferential directions for the semi-spheroid forming the outer layer 10. These circumferential directions are locally tangent to the semi-spheroid. The tapered struts 26 belonging to the subset 29 are oriented along a direction which is overall parallel to the meridian plane of the spheroidal outer layer 10.

In addition, the struts 26 of the subset 27 have their tapered shapes which are oriented along a direction D26 that is parallel to the equatorial plane P10 of the semi-spheroid forming the outer layer 10.

As shown in FIGS. 6 and 7, each strut 25 or each tapered strut 26 has overall a cylinder shape of which the axis connects two consecutive nodes 24 of the network 20. Each strut 25 or each tapered strut 26 has a cross-section that is oblong and symmetrical with respect to its longitudinal axis. Each strut 25 or tapered strut 26 has an overall pear-shaped cross-section.

As shown in FIGS. 1, 2, 7, 8 and 9, the outer layer 10 comprises several angularly juxtaposed networks 20. As shown in FIGS. 7 and 9, meshes 22.1 and 22.2 respectively belonging to two consecutive networks 20 form a dihedral angle A22 of about 25°.

Each network 20 substantially covers a quarter of the semi-spheroid forming the outer layer 10. Each quarter extends between meridians M1, M2 and the like which are spaced apart in pairs at an angle A22 of about 15°.

During computer-aided design (CAD) of the cup 2, each network 20 and the like is associated with a portion of the cup 2. In practice, a portion is repeated by rotation so as to design the entire cup 2. After manufacture of the cup 2 according to a method in accordance with the invention, the networks 20 and the like are almost imperceptible to the naked eye on the achieved cup 2. However, the networks 20 can be observed on the cup 2 by means of an optical magnifying instrument, for example a microscope.

FIGS. 10 and 11 illustrate a part of a cup in accordance with a second embodiment of the invention. Insofar as this cup is similar to the cup 2, the description of the cup 2 given hereinabove in connection with FIGS. 1 to 9 can be transposed to the cup of FIGS. 10 and 11, with the notable exception of differences set out hereinafter.

An element of the cup of FIGS. 10 and 11 that is identical or corresponding, by its structure or function, to an element of the cup 2 bears the same reference number incremented by 100. An outer layer 110, networks 120.1 and 120.2, meshes 122.1 and 122.2, nodes 124, cylindrical struts 125 with a circular base and tapered struts 126 with sharp edges 126.1 are thus defined.

As shown in FIG. 10, the cup of FIGS. 10 and 11 differs from the cup 2, as the outer layer 110 comprises two networks 120.1 and 120.2 which are interpenetrating and the meshes 122.1 and 122.2 of which have equivalent dimensions. In other words, the meshes 122.1 and 122.2 are stacked and shifted with identical orientations between meshes 122.1 and 122.2 of the networks 120.1 and 120.2.

A method in accordance with the invention allows making a cup in accordance with the invention, including the cup 2. Such a method comprises the steps of:
forming a powder stratum of the metallic material;
implementing a generative method machine, not shown, so as to sinter the stratum in a determined manner by a control unit, not shown;
repeating the two aforementioned steps until the cup 2 is formed.

The generative method machine can for example be a selective laser sintering machine that may process a metallic material, for example a machine produced by the companies PHENIX SYSTEM, EOS etc. Alternatively, the machine may implement a technique called electron beam melting technique.

The present invention has been exemplified hereinabove in relation to the embodiments illustrated in the figures. However, it is obvious that the present invention is not limited to these embodiments. On the contrary, the present invention comprises all technical equivalents of the described means as well as their technically possible combinations.

1. A cup, for orthopedic implant such as a cotyloidal implant, intended to be implanted in a bone, the cup having an inner cavity adapted to accommodate an articulation organ, the cup having an outer layer intended to be secured to the bone, the outer layer having overall the shape of a portion of a spheroid, the outer layer being made of metallic material;
wherein the outer layer comprises at least one network of meshes defined by nodes and by struts connecting the nodes together, each node being formed by the intersection of several struts, said struts comprising struts called tapered struts which each have a tapered shape, said tapered struts being arranged such that the tapered shapes are uniformly oriented.

2. The cup according to claim 1, wherein said tapered struts are arranged such that their tapered shapes are oriented along respective directions which are circumferential directions for the portion of the spheroid.

3. The cup according to claim 2, wherein at least one subset of tapered struts has tapered shapes oriented along a direction parallel to the equatorial plane of the portion of the spheroid.

4. The cup according to claim 1, wherein at least two tapered struts converge at each node.

5. The cup according to claim 1, wherein each strut has overall the shape of a cylinder of which the axis connects two consecutive nodes of said at least one network.

6. The cup according to claim 1, wherein each tapered strut has a cross-section that is oblong and symmetrical with respect to its longitudinal axis, each tapered strut preferably having an overall pear-shaped cross-section.

7. The cup according to claim 1, wherein the outer layer comprises several juxtaposed networks, meshes respectively belonging to two consecutive networks forming a dihedral angle of less than 35°.

8. The cup according to claim 7, wherein each network substantially covers a quarter of the portion of the spheroid, each quarter extending between the meridians spaced apart by an angle of less than 35°.

9. The cup according to claim 1, wherein the outer layer comprises two networks of meshes which are interpenetrating and the meshes of which have equivalent dimensions.

10. The cup according to claim 1, wherein each mesh has dimensions ranging between 200 micrometers and 800 micrometers.

11. The cup according to claim 1, wherein each mesh has overall a parallelepiped shape with a rectangular base, each mesh having a cube shape.

12. The cup according to claim 1, wherein the density of the outer layer is ranging between 30% and 90%.

13. The cup according to claim 1, wherein the outer layer has a thickness ranging between 0.3 mm and 7 mm.

14. The cup according to claim 1, wherein the metallic material is a material that is biocompatible, implantable and compatible with a generative method by powder sintering, the metallic material being selected from the group consisting of pure titanium, a titanium, chromium, cobalt, and stainless steel based alloy.

15. An orthopedic implant such as a cotyloidal implant, comprising a cup according to claim 1 and an articulation organ formed by an insert attached within the inner cavity.

16. A method for making a cup according to Claim 1, the method comprising the steps of:
forming a powder stratum of the metallic material;
implementing a generative method machine, so as to sinter the stratum in a determined manner by a control unit;
repeating the aforementioned steps until the cup is formed.
